(19) United States
(12) Patent Application Publication
(10) Pub. No.: US 2007/0073338 A1
(43) Pub. Date: Mar. 29, 2007

(54) Supercutaneous method and device for promoting hemostasis in cannulated patient

(76) Inventor: Roy Raghunandan, Suffolk County, NY (US)

Correspondence Address: MENDELSOHN AND ASSOCIATES, P.C., 1500 JOHN F. KENNEDY BLVD., SUTIE 405, PHILADELPHIA, PA, 19102, US

(21) Appl. No.: 10/570,691

(22) PCT Filed: Sep. 7, 2004
(86) PCT No.: PCT/US04/28724

§ 371 Date: Dec. 11, 2006

Related U.S. Application Data
(60) Provisional application No. 60/500,001, filed on Sep. 4, 2003.

(51) Int. Cl. A61B 17/08

(52) U.S. Cl. 606/215

(57) ABSTRACT

A supercutaneous hemostatic method and device reduces or eliminates bleeding while a bodily lumen is cannulated. The method involves wrapping a string (3) around a needle (2) protruding from the skin of the patient at a puncture site, pulling the ends of the string (3) in substantially opposing directions so that the ends of the string (3) overlap one another, forming a snug loop surrounding the needle (2), pulling the ends of the string substantially taut securing them to the skin of the patient, causing the loop to exert downward epidermal pressure at the puncture site to promote and facilitate hemostasis by means of applying a gentle, even compression. A device (50) for promoting hemostasis comprises a member having a central portion (51) engageable around an instrument protruding from a puncture site in the epidermis of a patient, and at least one projection (52) from the central portion

The present invention relates to the field of hemostatic devices; that is, medical instruments designed to stanch the flow of blood from a punctured blood vessel. More particularly, the present invention relates to a supercutaneous hemostatic method and device for use at a puncture site while a blood vessel is cannulated, without requiring further penetration of the skin.

Many medical procedures, including both therapeutic and diagnostic procedures, require access between two contiguous tissue samples, such as through the skin and into the vascular system of the patient. For example, although various means may be used to obtain access into a vein or artery, typically access is obtained by inserting either a needle, a cannula, a catheter (called an introducer catheter or sheath), or a stylet through the skin and into the selected blood vessel. A medical or diagnostic instrument, such as a guide wire, guiding catheter, balloon angioplasty device, atherectomy device, or the like is then inserted into the vascular system through the introducer catheter. In each event, the closing and subsequent healing of the resultant vascular puncture is critical to the successfull completion of the procedure. Additionally, a risk of hematoma exists, since bleeding from the vessel is likely to continue until sufficient clotting effects hemostasis.

Consequently, various devices have been developed for promoting hemostasis directly at the site of the vascular perforation. For example, there are devices that deploy intraluminal plugs within the vessel to close the puncture site, as disclosed in U.S. Pat. Nos. 4,852,568, 4,890,612, and 5,061,274 to Kensey, and U.S. Pat. No. 5,021,059 to Kensey et al. Another approach is to deliver a tissue adhesive or clotting agent to the perforation site, as disclosed in U.S. Pat. Nos. 5,221,259 and 5,419,765 to Weldon et al., U.S. Pat. No. 5,383,899 to Hammerslag and U.S. Pat. No. 5,486,195 to Myers et al. Still another approach is the application of pressure directly to the perforation site, as exemplified by PCT International Publication Number WO 95/32671, U.S. Pat. No. 4,619,261 to Guerrieo, and U.S. Pat. No. 4,929,246 to Sinofsky. Yet another approach is disclosed in U.S. Pat. No. 5,275,616 to Fowler, wherein a cylindrical plug is inserted along the shaft of a catheter segment extending from the skin surface to the blood vessel. The catheter is then removed so that the plug can expand as fluid is drawn into the plug from the vessel and the surrounding tissue. Unless pressure is applied, however, bleeding may occur around the plug into the subcutaneous tissue. A similar concept is disclosed in U.S. Pat. No. 5,391,183 to Janzen et al., which discloses a variety of plug delivery devices, including threaded plug pushers and multilegged channels, that install a plug that may be resorbable.

All of the above-noted devices are for hemostasis at a closed puncture site after removal of a needle, catheter, cannula, or stylet. Other percutaneous vascular hemostatic devices include mechanisms for delivering a suture percutaneously to a vascular suturing site, and then tying the suture in situ, such as U.S. Pat. Nos. 5,417,699 and 5,527,322 to Klein et al., U.S. Pat. No. 5,462,561 to Voda, and U.S. Pat. No. 5,364,408 to Gordon. While such devices, if properly employed, are capable of very effectively stemming blood flow, they may require a relatively high degree of dexterity to be operated properly. Furthermore, they tend to be somewhat complex and expensive to manufacture, and thus are not practically employed as single use, disposable products. Consequently, sterilization may be required between uses to reduce the risk of infection, thereby increasing their cost and inconvenience.

Most notably, however, is the fact that none of the aforementioned hemostatic devices are suitable for use while the patient is cannulated, i.e., while a needle, catheter, cannula, or stylet is still in the blood vessel of a patient. Such is the case with hemodialysis, wherein persons suffering kidney impairment cannot maintain a clean blood supply and must have their blood supply cleansed by an external machine. In such procedures, a dialyzer (artificial kidney) is temporarily integrated into the blood circulatory system to remove blood from a vein, i.e., blood carrying impurities from body organs and tissues, filter that blood within the artificial kidney machine, and return the cleansed blood to the circulation system at an artery for delivery to the body organs and tissues in the normal course. Thus, to perform dialysis the patient's blood system is accessed at two locations, i.e., to take and return blood. Such access is accomplished by inserting needles, catheters, cannulae or stylets through the skin and into the blood circulatory system. For most dialysis patients, however, the repeated access to the blood system by needles would damage the veins and arteries. To reduce such damage, surgery is typically performed to create a graft, fistula or shunt under the skin surface and in series with the blood system to allow frequent access by needle to the blood system without repeatedly puncturing the patient's veins and arteries. Control of puncture site bleeding after removal of the needles, catheters, cannulae, or stylets is usually accomplished by the application of external pressure to the skin entry site, e.g., using the fingers of trained medical personnel or the patient, or using any one of a number of well-known devices, such as the aforementioned devices, as well as others that simulate the required manual digital pressure in aid of hemostasis.

However, bleeding while a patient is cannulated is a problem that is not easily resolved and typically requires withdrawal of the needle(s), catheter(s), cannula(e), or stylet(s) and their subsequent reintroduction at the same or at an alternative puncture site. The typical dialysis patient requires dialysis two to three times per week, the total duration of which can be anywhere from 5 to 30 hours per week. Similarly, patients undergoing chemotherapy or infusion treatments and patients with continuous insulin infusion pumps can spend countless hours each week cannulated with one or more needles, catheters, cannulae, or stylets. For patients experiencing frequent puncture site bleeding, a number of various dressings, pads and bandages, including those using polyvinyl film, are frequently used to aid in hemostasis while a patient is cannulated. Many of these devices either must be applied prior to cannulation or else require removal and reintroduction of the needle, catheter, cannula, or stylet before they can be used. Further, many such devices suffer from an inability to provide sufficient external pressure at a cannulated puncture site to stop bleeding effectively, and therefore, a large number of patients still eventually require needle, catheter, cannula, or stylet reintroduction and must still needlessly suffer repeated episodes of puncture site bleeding, despite their use of such devices.

Thus, there is a demonstrated need for a supercutaneous device that effectively promotes hemostasis while a patient is cannulated, can easily be used by a patient or by medical personnel, can be used once bleeding at the puncture site starts without requiring re-cannulation, can be manufactured inexpensively, and is disposable.

The present invention provides a supercutaneous hemostatic method and device capable of reducing or eliminating bleeding while a bodily lumen is cannulated and having particular utility during procedures such as dialysis, chemotherapy, or infusion.

The method of the invention, in one aspect, begins by passing one end of a string around a needle protruding from the patient at a puncture site, bringing both ends of the string to one side of the needle. Next, the ends of the string are pulled in substantially opposing directions, so that a tightened and snug loop surrounding the needle is created, and the ends of the string overlap one another. The ends of the string are pulled substantially taut and are secured to the skin of the patient using fixation pads. When the ends of the string are fixed to the skin of the patient, the loop surrounding the needle exerts epidermal pressure at the puncture site in a downward direction, sufficient to promote and facilitate hemostasis by means of applying a gentle, even compression. In addition to the pressure exerted by the string, if the string is fabricated from or contains an absorbent material, the string may additionally aid in absorbing blood and promoting the formation of a clot around the needle, thereby “sealing”0 the puncture site. The invention prolongs access life at an access site by reducing the number of times re-cannulation must occur when bleeding at the access site begins.

FIG. 1 is a side perspective view illustrating the arm of a patient with a needle disposed therein;

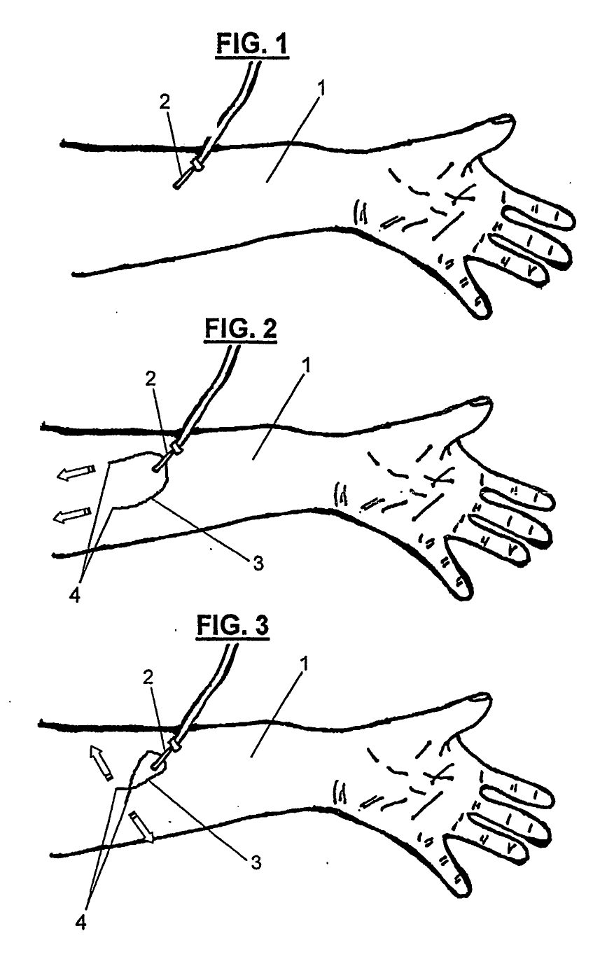

FIGS. 2-5 are side perspective views illustrating successive steps of an exemplary hemostasis method consistent with a first embodiment of the invention, wherein a string is utilized as an exemplary hemostatic device;

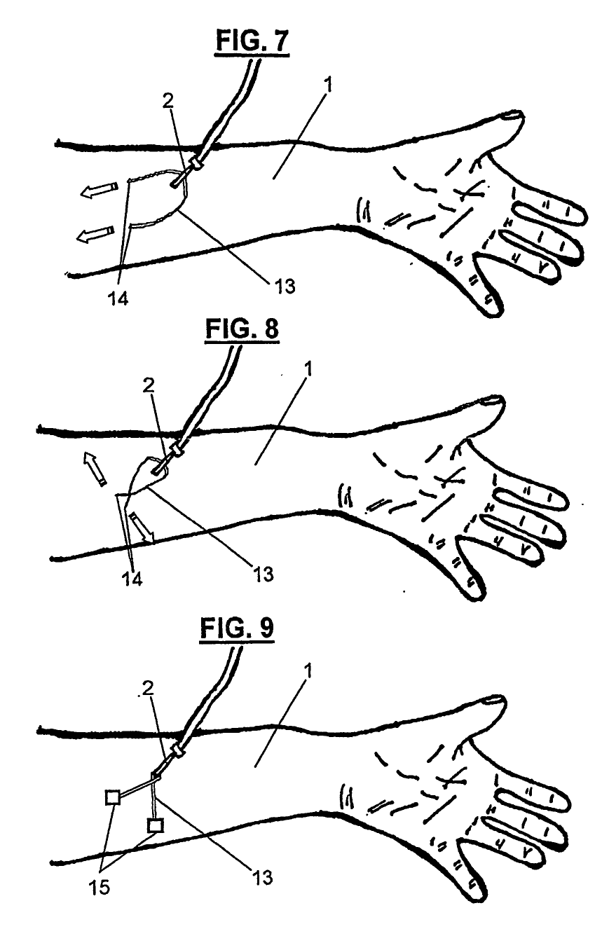

FIGS. 7-9 are side perspective views illustrating successive steps of an exemplary hemostasis method consistent with a second embodiment of the invention, wherein a flat strip is utilized as an exemplary hemostatic device;

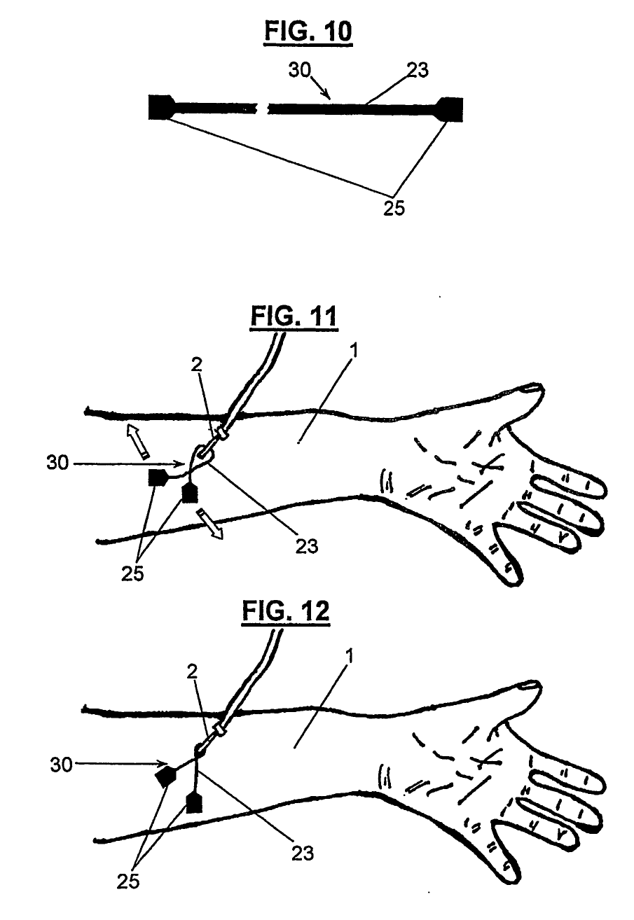

FIG. 10 is a plan view of an exemplary hemostatic device consistent with a third embodiment of the invention;

FIGS. 11 and 12 are side perspective views illustrating successive steps of an exemplary hemostasis method consistent with a third embodiment of the invention, wherein the exemplary hemostatic device of FIG. 10 is utilized;

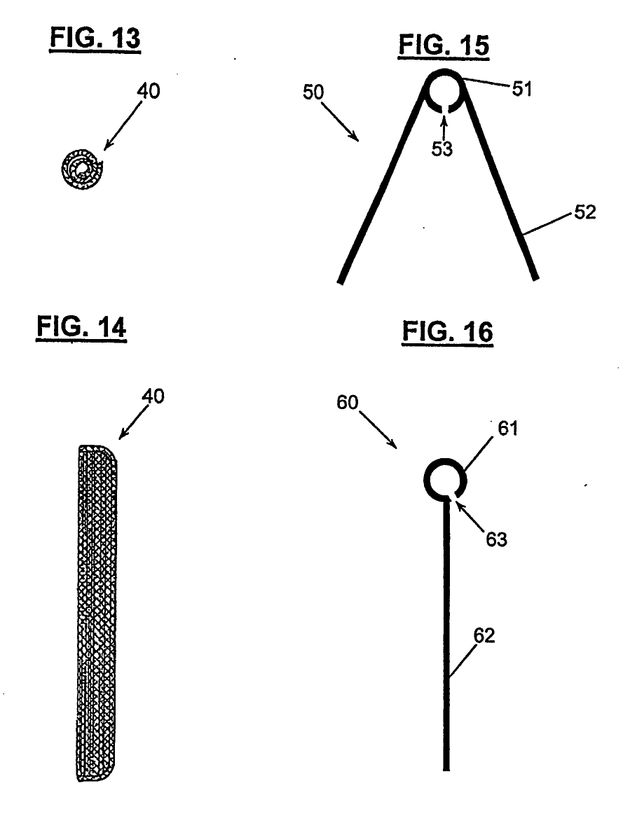

FIGS. 13 and 14 illustrate respectively end and top views of an alternative elongate member for use in an exemplary hemostasis method consistent with the present invention;

FIG. 15 is a plan view of an exemplary hemostatic device consistent with a fourth embodiment of the invention; and

FIG. 16 is a plan view of an exemplary hemostatic device consistent with a fifth embodiment of the invention.

Turning now to FIG. 1, the arm 1 of a patient with a needle 2 disposed therein at a puncture site in the epidermis is illustrated. While hemodialysis is the exemplary procedure for which the needle 2 is being used in the illustration, the procedure could be any requiring subcutaneous access, e.g., chemotherapy or continuous insulin infusion, and the needle 2 may alternatively be a catheter, cannula, stylet, or other subcutaneous access device.

With reference now to FIGS. 2-6, an exemplary hemostasis method consistent with a first embodiment of the invention is illustrated. In the embodiment shown in FIGS. 2-6, a string 3 having a diameter of approximately 1/16 inch and approximately 6 inches long is utilized as an exemplary hemostatic device.

As illustrated in FIG. 2, the first step of the method is to pass one end of the string 3 around the needle 2, bringing both ends 4 of the string 3 to one side of the needle 2.

Next, as FIG. 3 illustrates, the ends 4 of the string 3 are pulled in substantially opposing directions, so that a loop surrounding the needle 2 is created, and the ends 4 of the string 3 overlap one another.

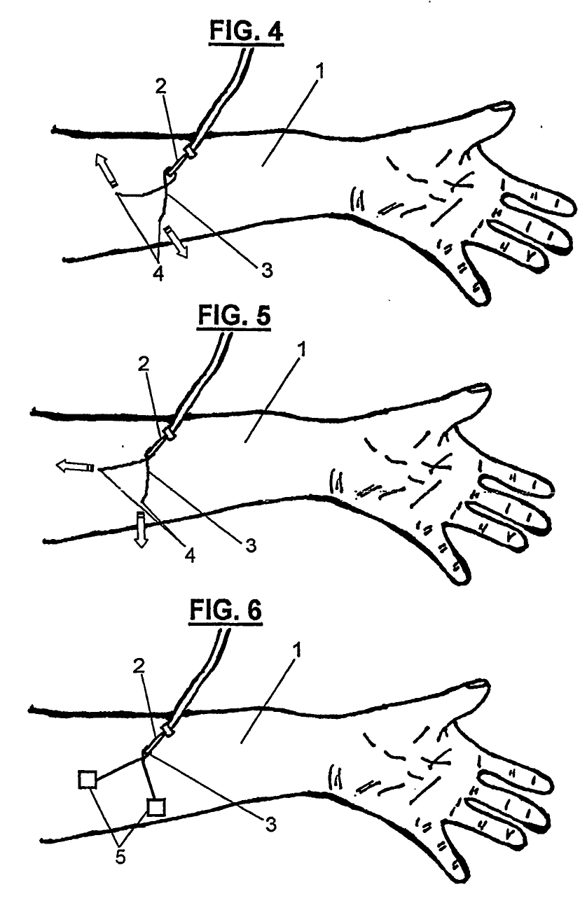

As shown in FIG. 4, the pulling of the ends 4 of the string 3 in substantially opposing directions is continued, to tighten the loop around the needle 2.

Turning now to FIG. 5, the pulling of the ends 4 of the string 3 continues until the loop is snug around the needle 2.

Finally, with reference to FIG. 6, when the ends 4 of the string 3 are pulled substantially taut, the ends 4 of the string 3 are secured to the epidermis of the arm 1 of the patient using fixation pads 5, which may be formed, e.g., of adhesive tape. It should be recognized that the pads 5 could be almost any fixation means, including adhesive-backed paper or cloth tape, a spot of glue, a polymer adhesive, or even non-adherent means, such as one or more straps, bands, or lengths of material such as self-adherent gauze (not shown) disposed around the arm 1 of the patient to which the ends 4 of the string 3 are affixed. The string 3 itself could be dimensioned to wrap entirely around the arm 1 of the patient and fasten to itself (not shown) or to the epidermis, e.g., on the side of the arm 1 opposite the puncture site. Alternatively, a single pad or section of tape or other adhesive material could be dimensioned large enough to fix both ends 4 of the string 3 to the epidermis of the patient at any location on the arm 1. The adhesive, adhesive pad 5, or other fixation means may be at the very end of the string 3, as shown in FIG. 6, or could be located anywhere between the needle 2 and the very end of the string 3. In particular, a “pigtail” of string 3 may extend beyond the pad 5 or other fixation means to permit easy removal of the pad 5 without moving or otherwise disturbing the needle 2.

When the ends 4 of the string 3 are fixed to the arm 1 of the patient, the loop surrounding the needle 2 exerts epidermal pressure at the puncture site in a downward direction, i.e., in a direction generally parallel to the longitudinal axis of the needle 2. The downward pressure exerted by the string 3 at the entry site of the needle 2 is sufficient to promote and facilitate hemostasis by means of applying a gentle, even compression, which can both reduce and prevent bleeding from the puncture site.

Moreover, in addition to the pressure exerted by the string 3, if the string 3 is fabricated from or contains an absorbent material, e.g., cotton, cloth, or gauze, the string 3 may additionally aid in absorbing blood and promoting the formation of a clot around the needle 2, thereby “sealing” the puncture site. In this scenario, the string 3 may be impregnated with a topical clotting agent such as bismuth subgalate or other coagulant solution, to aid in the clot formation.

Thus, the unique compressive and sealing properties of the string 3 disposed around the needle 2 at the puncture site by means of the fixation pads 5 provide an inexpensive hemostatic device that can be constructed or improvised from a variety of materials, as discussed herein. Advantageously, the use of a string 3 and pads 5 in this way prolongs access life an access site by reducing the number of times re-cannulation must occur when bleeding at the access site begins.

It should be noted that the angle of the needle 2 may vary, depending on a number of different factors and circumstances, and that the direction in which the ends 4 of the string 3 are initially pulled (as shown in FIG. 2) is preferably in the same direction or in a similar direction to that in which the needle 2 is inserted, although sufficient promotion of hemostasis through compression and absorption may be provided regardless of any particular direction in which the needle 2 and string 3 are disposed with respect to the patient.

With reference now to FIGS. 7-9, an exemplary hemostasis method consistent with a second embodiment of the invention is illustrated. In the embodiment shown in FIGS. 7-9, a section of tape 13, e.g., surgical tape consisting of a woven acetate-taffeta fabric coated with a layer of an acrylic adhesive, the tape 13 having a width of approximately 1/16 inch and being approximately 6 inches long, is utilized as an exemplary hemostatic device.

As illustrated in FIG. 7, the one end of the tape 13 is first passed around the needle 2, bringing both ends 14 of the tape 13 to one side of the needle 2, and pulling the ends 14 of the tape 13 in substantially opposing directions, so that a loop surrounding the needle 2 is created, and the ends 14 of the tape 13 overlap one another.

As shown in FIG. 8, the pulling of the ends 14 of the tape 13 in substantially opposing directions is continued, to tighten the loop around the needle 2, and the pulling of the ends 14 of the tape 13 continues until the loop is snug around the needle 2.

Finally, with reference to FIG. 9, when the ends 14 of the tape 13 are pulled substantially taut, the ends 14 of the tape 13 are secured to the arm 1 using the adhesive disposed along the underside of the tape 13. Additionally, fixation pads 15, which may be provided to increase the adhesion of the tape 13 to the skin of the arm 1 of the patient, may be formed, e.g., of adhesive tape or another fixation means, including adhesive-backed paper or cloth tape, a spot of glue, a polymer adhesive, or even non-adherent means, such as one or more straps, bands, or lengths of material such as self-adherent gauze (not shown) disposed around the arm 1 of the patient to which the ends 14 of the tape 13 are affixed. The tape 13 itself could be dimensioned to wrap entirely around the arm 1 of the patient and fasten to itself (not shown) or to the epidermis, e.g., on the side of the arm 1 opposite the puncture site. Alternatively, a single fixation pad or section of tape or other adhesive material could be dimensioned large enough to fix both ends 14 of the tape 13 to the epidermis of the patient The adhesive, adhesive pad 15, or other fixation means may be at the very end of the tape 13, as shown in FIG. 9, or could be located anywhere between the needle 2 and the very end of the tape 13. In particular, a “pigtail” of tape 13 could extend beyond the pad 15 or other fixation means to permit easy removal of the pad 15 without moving or otherwise disturbing the needle 2.

When the ends 14 of the tape 13 are fixed to the arm 1 of the patient, the loop surrounding the needle 2 exerts epidermal pressure at the puncture site in a downward direction, i.e., in a direction generally parallel to the longitudinal axis of the needle 2. The downward pressure exerted by the tape 13 at the entry site of the needle 2 is sufficient to promote and facilitate hemostasis by means of applying a gentle, even compression, which can both reduce and prevent bleeding from the puncture site.

It should be noted that the use of tape or other elongate member having adhesive properties along its entire length, consistent with the second embodiment of the invention, may provide certain additional advantages over string or another elongate member having no adhesive properties along its length and only fastened at its ends. Specifically, the adhesive along the length of the elongate member aids in fixing the elongate member to the needle, catheter, cannula, or stylet around which it is wrapped, as well as in fixing the ends of the elongate member that project away from the puncture site to the skin of the patient along the entire length of the elongate member, potentially providing an increase in compressive force close to the puncture site.

Moreover, in addition to the pressure exerted by the tape 13, if the tape 13 is fabricated from or contains an absorbent material, e.g., cotton, cloth, or gauze, the tape 13 may additionally aid in absorbing blood and promoting the formation of a clot around the needle 2, thereby “sealing” the puncture site. In this scenario, the string 3 may be impregnated with a topical clotting agent such as bismuth subgalate or other coagulant solution, to aid in the clot formation.

With reference now to FIGS. 10-12, an exemplary hemostatic device 30 and its method of use consistent with a third embodiment of the invention are illustrated.

As shown in FIG. 10, the hemostatic device 30 includes string- or tape-like material, e.g., surgical tape consisting of a non-extensible woven acetate-taffeta fabric coated with a layer of an acrylic adhesive, and has an elongate body 23 disposed between a pair of adhesive pad portions 25. In the exemplary embodiment shown, the body 23 is flat and has a width of approximately 1/16 inch and is approximately 6 inches long. The pad portions 25 may be formed, e.g., of adhesive tape or another fixation means, including adhesive-backed paper or cloth tape, glue, or a polymer adhesive, and the pad portions 25 may be formed from the same material as the body 23 of the device 30, or alternatively, may be formed from a different material. For example, in an embodiment in which the body 23 of the device 30 is simply a cotton string, the pad portions 25 might include bioadhesive tape. It should be noted that, while in the embodiment illustrated, the pad portions 25 are pentagonal in shape, any size or shape pads could alternatively be employed to serve a substantially similar function, and the pads may not even be necessary, if the body 23 of the device 30 is dimensioned to wrap entirely around the arm 1 of the patient and fasten to itself (not shown) or to the epidermis, e.g., on the side of the arm 1 opposite the puncture site, or if the body 23 of the device 30 includes adhesive tape or another fixation means, including adhesive-backed paper or cloth tape, glue, or a polymer adhesive, and the pad portions. Alternatively, a single pad or section of tape or other adhesive material could be dimensioned large enough to fix both ends 25 of the device 30 to the epidermis of the patient at any location on the arm 1. The pad 25 or other fixation means may be at the very end of the body 23 of the device 30, as shown in FIG. 10, or could be disposed so as to form a “pigtail” of non-adhesive portion of the body 23 of the device 30 that extends beyond the pad 25 or other fixation means to permit easy removal of the pad 25 without moving or otherwise disturbing the needle 2.

As illustrated in FIG. 11, one end of the device 30 is first passed around the needle 2, bringing both pads 25 of the device 30 to one side of the needle 2, and pulling the pads 25 of the device 30 in substantially opposing directions, so that the body 23 forms a loop surrounding the needle 2, with the body 23 of the device 30 overlapping itself. The pulling of the pads 25 of the device 30 in substantially opposing directions is continued, to tighten the loop around the needle 2, and the pulling of the pads 25 of the device 30 continues until the loop is snug around the needle 2.

With reference to FIG. 12, when the pads 25 of the tape 30 are pulled so that the body 23 of the device 30 is substantially taut, the pads 25 are secured to the arm 1 by means of the adhesive on the pads 25.

When the pads 25 are fixed to the arm 1 of the patient, the loop of the body 23 of the device 30 surrounding the needle 2 exerts epidermal pressure at the puncture site in a downward direction, i.e., in a direction generally parallel to the longitudinal axis of the needle 2. The downward pressure exerted by the device 30 at the entry site of the needle 2 is sufficient to promote and facilitate hemostasis by means of applying a gentle, even compression, which can both reduce and prevent bleeding from the puncture site.

Moreover, in addition to the pressure exerted by the device, if the body 23 of the device 30 is fabricated from or contains an absorbent material, e.g., cotton, cloth, or gauze, the tape 13 may additionally aid in absorbing blood and promoting the formation of a clot around the needle 2, thereby “sealing” the puncture site. In this scenario, the body 23 of the device 30 may be impregnated with a topical clotting agent such as bismuth subgalate or other coagulant solution, to aid in the clot formation.

FIGS. 13 and 14 illustrate respectively end and top views of an alternative elongate member 40 for use in the method of the present invention. In the embodiment shown, a square, rectangular, or other shaped flat section of material 40, e.g., gauze, is rolled to form an elongate member having increased strength and/or absorbency properties and is then used in place of a string, tape, or custom device as described in the first through third embodiments hereinabove.

FIG. 15 illustrates an exemplary hemostatic device 50 consistent with a fourth embodiment of the invention. In this embodiment, the device 50 is embodied as a unitary member having an apertured portion 51 and two (or more) elongated portions 52. By means of an opening 53 in the apertured portion 51, the apertured portion 51 of the device 50 is applied around a needle, cannula, or other access device projecting from the skin of the patient, and the elongated portions 52 are secured to the skin of the patient by adhesive pads or another of the methods described herein, so that the apertured portion 61 of the device 60 exerts downward pressure at the entry site of the access device, sufficient to promote and facilitate hemostasis by means of applying a gentle, even compression, which can both reduce and prevent bleeding from the puncture site. The apertured portion 63 may be sized to fit snugly around the access device, or may be spaced so that only part of the apertured portion 63 makes contact with the access device. The provision of an opening 53 in the apertured portion 51 is not necessary if the device 50 is pre-installed by passing the needle, cannula, or other access device through the apertured portion 51 prior to insertion of the needle, cannula, or other access device into the skin of the patient. Alternatively, the device 50 could be integrated into a needle or cannula assembly, or provided as part of a kit.

FIG. 16 illustrates an exemplary hemostatic device 60 consistent with a fifth embodiment of the invention. In this embodiment, the device 60 is embodied as a unitary member having an apertured portion 61 and only a single elongated portion 62. By means of an opening 63 in the apertured portion 61, the apertured portion 61 of the device 60 is applied around a needle, cannula, or other access device projecting from the skin of the patient, and the single elongated portion 62 is secured to the skin of the patient by an adhesive pad or another of the methods described herein, so that the apertured portion 61 of the device 60 exerts downward pressure at the entry site of the access device, sufficient to promote and facilitate hemostasis by means of applying a gentle, even compression, which can both reduce and prevent bleeding from the puncture site. The apertured portion 63 may be sized to fit snugly around the access device, or may be spaced so that only part of the apertured portion 63 makes contact with the access device. The provision of an opening 63 in the apertured portion 61 is not necessary if the device 60 is pre-installed by passing the needle, cannula, or other access device through the apertured portion 61 prior to insertion of the needle, cannula, or other access device into the skin of the patient. Alternatively, the device 60 could be integrated into a needle or cannula assembly, or provided as part of a kit.

It should be recognized that, although the method of the invention is described hereinabove as using a string, tape, or custom device as a hemostatic device, other components may additionally or alternatively be employed to perform substantially the same function, e.g., a cord, a rope, a strand, a strip, a wire, a thread, a braid, a line, a bandage, a belt, a binding, a cable, a chain, a fillet, a ligature, a ribbon, a strap, a tie, a truss, a lanyard, elastic, twine, or a section of fabric or other material, and the component used may or may not be flexible. Other shapes for such components are also contemplated, and a device consistent with the invention should not be construed as limited to including components having elongate, string-like, or tape-like shapes.

It should further be understood that the dimensions of the exemplary string, tape, and custom device described above (i.e., length and width/diameter) are provided purely by way of example, and a device consistent with the invention may be constructed so as to have dimensions other than those set forth hereinabove.

It should be recognized that, while the invention is described hereinabove as used at a puncture site on the arm of a patient, a method or device consistent with the present invention may alternatively be used at another location on the body of a patient. Moreover, while the invention is described hereinabove as having particular utility with needles, catheters, cannulae, and stylets, it should be understood that the invention may be used with other medical or diagnostic devices (e.g., introducers, scopes, or other vascular access devices) protruding from a puncture site, to promote hemostasis at that site.

Further, while a string, tape, or custom device consistent with the invention is described hereinabove as being impregnable with a topical clotting agent, other compositions may alternatively or additionally be implanted therein, e.g., topical antibiotics, antiseptics, anesthetics, and other such medicaments. A device consistent with the invention may also include a hydrocolloid adhesive composition having an anti-microbial agent, e.g., one that includes metallic compounds that provide anti-microbial effects by killing bacteria from contact with metallic ions.

Although the present invention has been set forth in terms of the embodiments described herein, it is to be understood that such disclosure is purely illustrative and is not to be interpreted as limiting. Consequently, without departing from the spirit and scope of the invention, various alterations, modifications, and/or alternative applications of the invention will, no doubt, be suggested to those skilled in the art after having read the preceding disclosure. Accordingly, it is intended that the present invention be interpreted as encompassing all alterations, modifications, or alternative applications as fall within the true spirit and scope of the invention.

1-29. (canceled)

30. A method of promoting hemostasis, the method comprising:
using a member to apply pressure to epidermis of a patient in a region surrounding an instrument protruding from a puncture site in the epidermis, wherein the member:
(1) has an elongated portion that substantially surrounds the instrument, and
(2) has one or more portions thereof secured to the epidermis of the patient.

31. A method of promoting hemostasis, the method comprising:
wrapping an elongated device around an instrument protruding from a puncture site in the epidermis of a patient, the elongated device having opposing ends;
pulling the ends of the elongated device in substantially opposing directions so that the ends of the elongated device overlap one another, thereby forming a loop surrounding the instrument; and
securing the ends to the epidermis of the patient.

32. A device for promoting hemostasis at a puncture site in the epidermis of a patient, the device comprising:
a member having:
(i) a central portion engageable around an instrument protruding from a puncture site in the epidermis of a patient; and
(ii) at least one projection from the central portion;
wherein at least a portion of the projection is detachably engageable to the epidermis of the patient in a region near the puncture site, so as to bias at least a region of the central portion toward the detachably engageable portion of the projection if:
(a) the central portion is engaged around the instrument protruding from the puncture site, and
(b) the detachably engageable portion of the projection is engaged to the epidermis of the patient.

33. A device as claimed in claim 32, wherein the detachably engageable portion of the projection comprises an adhesive material in at least one region thereof.

34. A device as claimed in claim 32, wherein the central portion is flexible.

35. A device as claimed in claim 32, wherein the central portion is elongated in one dimension and has a thickness in another dimension.

36. A device as claimed in claim 35, wherein the at least one projection is enlarged relative to the thickness of the central portion.

37. A device as claimed in claim 32, wherein the central portion comprises string or tape.

38. A device as claimed in claim 32, wherein the central portion comprises an absorbent material.

39. A device as claimed in claim 32, wherein the central portion comprises a clotting agent.

40. A device as claimed in claim 32, wherein the central portion comprises an antibiotic or antiseptic composition.

41. A device as claimed in claim 32, wherein the central portion comprises an anesthetic composition.

42. A device as claimed in claim 32, wherein the central portion comprises a hydrocolloid adhesive composition having an anti-microbial agent.

43. A device as claimed in claim 32, wherein the central portion comprises a non-extensible woven acetate-taffeta material.

44. A device as claimed in claim 32, further comprising an acrylic adhesive coating over at least a portion of the member.

45. A device as claimed in claim 32, wherein at least the central portion of the member comprises a rolled material forming an elongate member.

46. A device as claimed in claim 32, wherein the detachably engageable portion of the projection is at the very end of the projection.

47. A device for promoting hemostasis at a puncture site in the epidermis of a patient, the device comprising:
a member having:
a central portion engageable around an instrument protruding from a puncture site in the epidermis of a patient; and
opposing ends relative to the central portion;
the opposing ends being detachably engageable either to each other or to the epidermis of the patient, so as to bias the central portion to apply pressure on the epidermis in a region surrounding the instrument protruding from the puncture site if:
(a) the central portion is engaged around the instrument protruding from the puncture site, and
(b) the ends are engaged either to each other or to the epidermis of the patient.

48. A device as claimed in claim 47, wherein the central portion comprises string or tape.

49. A device as claimed in claim 47, wherein the central portion comprises a non-extensible woven acetate-taffeta material.
